(19) United States
(12) Patent Application Publication
(10) Pub. No.: US 2006/0147995 A1
(43) Pub. Date: Jul. 6, 2006

(54) Method for producing antibody fragments

(75) Inventors: Leo Gerardus Joseph Frenken, Vlaardingen (NL); Cornelis Paul Erik van der Logt, Vlaardingen (NL)

Correspondence Address: MORGAN LEWIS & BOCKIUS LLP, 1111 PENNSYLVANIA AVENUE NW, WASHINGTON, DC, 20004, US

(73) Assignee: Unilever Patent Holdings B.V.

(21) Appl. No.: 11/183,814

(22) Filed: Jul. 19, 2005

Related U.S. Application Data
(63) Continuation-in-part of application No. 09/626,242, filed on Sep. 27, 2000, now abandoned.

(30) Foreign Application Priority Data
Jan. 26, 1998 (EP) 98300525.7

(51) Int. Cl. C40B 40/10; C07K 16/44

(52) U.S. Cl. 435/7.1; 530/387.3

(57) ABSTRACT

An expression library comprising a repertoire of nucleic acid sequences each encoding at least part of a variable domain of a heavy chain derived from an immunoglobulin naturally devoid of light chains and its use in producing antibodies, particularly fragments thereof, is disclosed. The invention provides a method for preparing antibodies, or fragments thereof, having a specificity for a target antigen which avoids the need for the donor previously to have been immunised with the target antigen.

### FIELD OF THE INVENTION

The present invention relates to an expression library comprising a repertoire of nucleic acid sequences cloned from a non-immunised source, each nucleic acid sequence encoding at least part of a variable domain of a heavy chain derived from an immunoglobulin naturally devoid of light chains and its use in producing antibodies, or more particularly fragments thereof. In particular, the invention relates to a method for the preparation of antibodies or fragments thereof having binding specificity for a target antigen which avoids the need for the donor previously to have been immunised with the target antigen.

### BACKGROUND OF THE INVENTION

Monoclonal antibodies, or binding fragments thereof, have traditionally been prepared using hybridoma technology (Kohler and Milstein, 1975, Nature 256, 495). More recently, the application of recombinant DNA methods to generating and expressing antibodies has found favour. In particular, interest has concentrated on combinatorial library techniques with the aim of utilising more efficiently the antibody repertoire.

The natural immune response in vivo generates antigen-specific antibodies via an antigen-driven recombination and selection process wherein the initial gene recombination mechanism generates low specificity, low-affinity antibodies. These clones can be mutated further by antigen-driven hypermutation of the variable region genes to provide high specificity, high affinity antibodies.

Approaches to mimicking the first stage randomisation process which have been described in the literature include those based on the construction of ‘naive’ combinatorial antibody libraries prepared by isolating panels of immunoglobulin heavy chain variable (VH) domains and recombining these with panels of light variable chains (VL) domains (see, for example, Gram et al, Proc. Natl. Acad. Sa, USA, 89, 3576-3580, 1992). Naive libraries of antibody fragments have been constructed, for example, by cloning the rearranged V-genes from the IgM RNA of B cells of unimmunised donors isolated from peripheral blood lymphocytes, bone marrow or spleen cells (see, for example, Griffiths et al, EMBO Journal, 12(2), 725-734, 1993, Marks et al, J. Mol. Biol., 222, 581-597, 1991). Such libraries can be screened for antibodies against a range of different antigens.

In combinatorial libraries derived from a large number of VH genes and VL genes, the number of possible combinations is such that the likelihood that some of these newly formed combinations will exhibit antigen-specific binding activity is reasonably high provided that the final library size is sufficiently large. Given that the original B-cell pairing between antibody heavy and light chain, selected by the immune system according to their affinity of binding, are likely to be lost in the randomly, recombined repertoires, low affinity pairings would generally be expected. In line with expectations, low affinity antibody fragments (Fabs) with Kₐs of 10⁴-10⁵ M⁻¹ for a progesterone-bovine serum albumin (BSA) conjugate have been isolated from a small (5×10⁶) library constructed from the bone marrow of non-immunised adult mice (Gram et al, see above).

Antibody fragments of higher affinity (Kₐs of 10⁶-10⁷ M⁻¹ range) were selected from a repertoire of 3×10⁷ clones, made from the peripheral blood lymphocytes of two healthy human volunteers (Marks et al, see above) comprising heavy chain repertoires of the IgM (naive) class. These were combined with both Lamda and Kappa light chain sequences, isolated from the same source. Antibodies to more than 25 antigens were isolated from this library, including self-antigens (Griffiths et al, see above) and cell-surface molecules (Marks et al, Bio/Technology, 11, 1145-1149, 1993).

The second stage of the natural immune response, involving affinity maturation of the selected specificities by mutation and selection has been mimicked in-vitro using the technique of random point mutation in the V-genes and selecting mutants for improved affinity. Alternatively, the affinity of antibodies may be improved by the process of “chain shuffling”, whereby a single heavy or light chain is recombined with a library of partner chains (Marks et al, Bio/Technology, 10 779-782, 1992).

Recently, the construction of a repertoire of 1.4×10¹⁰ scFv clones, achieved by ‘brute force’ cloning of rearranged V genes of all classes from 43 non-immunised human donors has been reported (Vaughan et al 1996) and Griffiths et al, see above. Antibodies to seven different targets (including toxic and immunosuppressant molecules) were isolated, with measured affinities all below 10 nM.

The main limitation in the construction of combinatorial libraries is their size, which consequently limits their complexity. Evidence from the literature suggests that there is a direct link between library size and diversity and antibody specificity and affinity (see Vaughan et al, Nature Biotechnology, 14, 309-314, 1996), such that the larger (and more diverse) the library, the higher the affinity of the selected antibodies. On this basis, single domain libraries, which omit the process of recombination which is responsible for the generation of variability, would not be expected to be an effective source of high affinity and high specificity antibodies.

EP-B-0368684 (Medical Research Council) discloses the construction of expression libraries comprising a repertoire of nucleic acid sequences each encoding at least part of an immunoglobulin variable domain and the screening of the encoded domains for binding activities. It is stated that repertoires of genes encoding immunoglobulin variable domains are preferably prepared from lymphocytes of animals immunised with an antigen. The preparation of antigen binding activities from single VH domain, the isolation of which is facilitated by immunisation, is exemplified (see Example 6). Repertoires of amplified heavy chain variable domains obtained from mouse immunised with lysozyme and from human peripheral blood lymphocytes were cloned into expression vectors and probed for lysozyme binding activity. It is reported that 2 positive clones (out of 200) were identified from the amplified mouse spleen DNA and 1 clone from the human cDNA. A library of VH domains from the immunised mouse was screened for lysozyme and keyhole limpet haemocyanin (KLH) binding activities; from 2000 colonies, 21 supernatants were found to have lysozyme binding activity and 2 to have KLH binding activity. An expression library prepared from a mouse immunised with KLH screened in the same manner gave 14 supernatants with KLH binding activity and only 1 with lysozyme binding activity. These results suggest to the Applicants that although antigen binding activities can be seen, these are of very low specificity and affinity (presumably due to the absence of the stabilising effect of the missing light chain such that only half of the designed binding pocket is present, leading to binding with related or homologous targets).

Immunoglobulins capable of exhibiting the functional properties of conventional (four-chain) immunoglobulins but which comprise two heavy polypeptide chains and which furthermore are devoid of light polypeptide chains have been described (see European Patent Application EP-A-0584421, Casterman et al, 1994). Fragments of such immunoglobulins, including fragments corresponding to isolated heavy chain variable domains or to heavy chain variable domain dimers linked by the hinge disulphide are also described. Methods for the preparation of such antibodies or fragments thereof on a large scale comprising transforming a mould or yeast with an expressible DNA sequence encoding the antibody or fragment are described in patent application WO 94/25591 (Unilever).

The immunoglobulins described in EP-A-0584421, which may be isolated from the serum of Camelids, do not rely upon the association of heavy and light chain variable domains for the formation of the antigen-binding site but instead the heavy polypeptide chains alone naturally form the complete antigen binding site. These immunoglobulins, hereinafter referred to as “heavy-chain immunoglobulins” are thus quite distinct from the heavy chains obtained by the degradation of conventional (four-chain) immunoglobulins or by direct cloning. Heavy chains from conventional immunoglobulins contribute part only of the antigen-binding site and require a light chain partner, forming a complete antigen binding site, for optimal antigen binding.

As described in EP-A-0584421, heavy chain immunoglobulin VH regions isolated from Camelids (forming a complete antigen binding site and thus constituting a single domain binding site) differ from the VH regions derived from conventional four-chain immunoglobulins in a number of respects, notably in that they have no requirement for special features for facilitating interaction with corresponding light chain domains. Thus, whereas in conventional (four-chain) immunoglobulins the amino acid residue at the positions involved in the V_{H}V_{L} interaction is highly conserved and generally apolar leucine, in Camelid derived V_{H} domains this is replaced by a charged amino acid, generally arginine. It is thought that the presence of charged amino acids at this position contributes to increasing the solubility of the camelid derived V_{H}. A further difference which has been noted is that one of the CDRs of the heavy chain immunoglobulins of EP-A-0584421, the CDR₃, may contain an additional cysteine residue associated with a further additional cysteine residue elsewhere in the variable domain. It has been suggested that the establishment of a disulphide bond between the CDR₃ and the remaining regions of the variable domain could be important in binding antigens and may compensate for the absence of light chains.

cDNA libraries composed of nucleotide sequences coding for a heavy-chain immunoglobulin and methods for their preparation are disclosed in EP-A-0584421. However, EP-A-0584421 does not teach that libraries can be prepared from non-immunised animals or that an individual library can be used to identify antibodies to a range of different antigens to which the donor animal has not previously been exposed. On the contrary, the approach suggested in EP-A-0584421 is to pre-immunise the animal with an antigen of interest so that antibodies can be selected which have specificity for that antigen of interest. Further, no actual examples of the preparation of libraries or antibodies are given in the specification of EP-A-0584421, the sections related library and antibody preparation are entirely speculative with no experimental support given.

The need for prior immunisation is also referred to in Arabi Ghahroudi et al (FEBS Letters, 414 (1997), 521-526.

### SUMMARY OF THE INVENTION

In a first aspect, the invention provides an expression library comprising a plurality, such as a repertoire, of nucleic acid sequences cloned from a non-immunised source, each nucleic acid sequence encoding at least part of a variable domain of a heavy chain derived from an immunoglobulin naturally devoid of light chains.

Preferably the plurality of nucleic acid sequences comprises at least 10⁷ different sequences, more preferably at least 5×10⁷ different sequences, such as at least 10⁸ different sequences.

Further provided is a method of preparing a cDNA expression library as set forth above comprising providing mRNA, such as a repertoire of mRNA, from a non-immunised source, treating the obtained RNA with a reverse transcriptase to obtain the corresponding cDNA and cloning the cDNA, with or without prior PCR amplification, into an expression vector. Expression vectors comprising such nucleic acid sequences and host cells transformed with such expression vectors are also provided.

Typically the mRNA represents the repertoire of expressed immunoglobulins naturally devoid of light chains in the source organism from which the mRNA is derived e.g. the mRNA obtained from a population of lymphoid cells, such as B lymphocytes.

Further provided is the use of a non-immunised source of nucleic acid sequences encoding at least part of a variable domain of a heavy chain derived from an immunoglobulin naturally devoid of light chains to prepare an expression library.

In another aspect, the invention provides a method for the preparation of antibody fragments derived from a non-immunised source having specificity for a target antigen comprising screening an expression library as set forth above for antigen binding activity and recovering antibody fragments having the desired specificity.

In a particular embodiment, the present invention provides a method for selecting one or more antibody fragments derived from a non-immunised source having binding specificity for a target antigen, the method comprising

(i) screening an expression library with the target antigen, the library comprising a plurality of nucleic acid sequences cloned from a non-immunised source, each nucleic acid sequence encoding at least part of a variable domain of a heavy chain derived from an immunoglobulin naturally devoid of light chains, the plurality of nucleic acid sequences comprising at least 10⁷ different sequences;

(ii) selecting one or more antibody fragments having the desired specificity for the target antigen; and optionally

(iii) recovering the one or more antibody fragments having the desired binding specificity.

In one embodiment, the method further comprises a step (iv) of isolating the nucleic acid sequence(s) encoding the selected one or more antibody fragments.

The invention further provides the use of a non-immunised source of nucleic acid sequences encoding at least part of a variable domain of a heavy chain derived from an immunoglobulin naturally devoid of light chains to prepare an antibody, or fragment thereof, having binding specificity for a target antigen.

According to a further aspect, nucleic acid sequences encoding antibody fragments isolated from such a repertoire of variable region genes may be attached to nucleic acid sequences encoding one or more suitable heavy chain constant domains and expressed in a host cell, providing complete heavy chain antibodies.

In a particular embodiment, the present invention provides a method for preparing an antibody derived from a non-immunised source having binding specificity for a target antigen, the method comprising

(i) isolating a nucleic acid sequence encoding an antibody fragment having the desired binding specificity for the target antigen by the method described above, including step (iv); and

(ii) operably linking the region of the nucleic acid sequence encoding at least part of a variable domain of a heavy chain derived from an immunoglobulin naturally devoid of light chains to one or more nucleic acid sequences encoding one or more heavy chain constant domains; and

(iii) expressing the resulting product in a host cell.

By means of the invention, antibodies, particularly fragments thereof, having a specificity for a target antigen may conveniently be prepared by a method which does not require the donor previously to have been immunised with the target antigen. The method of the invention provides an advantageous alternative to hybridoma technology, or cloning from B cells and spleen cells where for each antigen, a new library is required.

The present invention may be more fully understood with reference to the following description, when read together with the accompanying drawings.

### BRIEF DESCRIPTION OF THE DRAWINGS

FIG. 1 shows a schematic representation of the domain structure of the ‘classical’ four-chain/two domain antibodies (a) and the camelid two chain/single domain antibodies (b).

FIG. 2 shows a plasmid map of phage display vector pHEN.5 containing a heavy chain variable domain (HC-V) gene. The DNA and protein sequences of the insertion regions are indicated.

FIGS. 3A, 3B show a specificity ELISA assay of HC-V-myc samples of clones selected by panning on RR6-BSA (1% gelatin block).
A Specific clones.
B ‘sticky’ aspecific clones.
RR-6 is an azo dye, available from ICI; BSA is bovine serum albumin; myc is a peptide comprising the sequence Glu-Gln-Lys-Leu-Ile-Ser-Glu-Glu-Asp-Leu-Asn.

FIG. 4 shows inhibition assays of HC-Vs selected by panning on RR6-BSA. Crude HC-V-myc samples were preincubated with increasing concentrations of RR6-BSA, followed by assay of free HC-V-myc on immobilised RR6-BSA.

FIG. 5 shows aligned protein sequences of selected anti-RR6 clones. The CDR regions are boxed.

FIG. 6 shows a specificity ELISA assay of HC-V-myc samples of clones selected by panning on Dicarboxylic linoleic acid—ovalbumin conjugate (Di-OVA) (1% gelatin block).

FIG. 7 shows inhibition of antigen binding activity of the anti-dicarboxylic acid clones D1, D2 and D3 by the presence of free target antigen (Di-OVA) or control conjugate (estrone 3-glucuronide, E3G-OVA).

FIG. 8 shows aligned protein sequences of the three selected anti-dicarboxylic clones D1, D2, D3. The CDR regions are boxed.

FIG. 9 shows the effect of ammonium thiocyanate (ATC) on binding of HC-Vs to immobilised RR6-BSA. Increasing concentrations of ATC were added to crude HC-V-myc samples bound to immobilised RR6-BSA, followed by detection of remaining bound HC-V using anti-myc monoclonal antibody.

FIG. 10 shows the effect of ATC on binding of HC-Vs to immobilised Di-OVA. Increasing concentrations of ATC were added to crude HC-V-myc samples bound to immobilised Di-OVA, followed by detection of remaining bound HC-V using anti-myc monoclonal antibody.

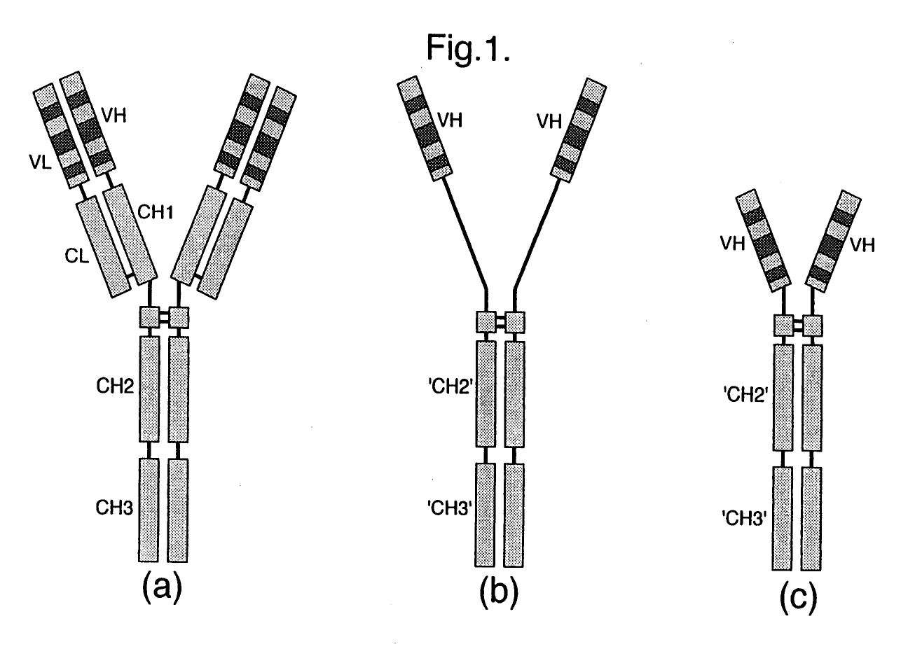

### DETAILED DESCRIPTION OF THE INVENTION

The invention is based on the unexpected finding that highly specific antibody fragments against a target antigen may be provided by screening an expression library comprising a repertoire of nucleic acid sequences, each encoding at least part of a variable domain of a heavy chain derived from a non-immunised source of an immunoglobulin naturally devoid of light chains, for antigen binding activity. It would not be predicted that single domain libraries would provide high affinity/high specificity antibodies (in the order of 10 to 100 nM) for the reasons of absence of combinatorial effect discussed above. From the teaching of EP-A-0584421, it would have been expected that in order to produce an antibody specific for a target antigen, either pre-immunisation of the donor with the target antigen or random combination with a VL domain would be necessary. Furthermore, we have found that a single library can be used to screen for high affinity antibodies to a range of different antigens.

As used herein, the term “antibody” refers to an immunoglobulin which may be derived from natural sources or synthetically produced, in whole or in part. An “antibody fragment” is a portion of a whole antibody which retains the ability to exhibit antigen binding activity, generally comprising one or more complementarity determining regions (CDRs).

The heavy chain variable domains for use according to the invention may be derived from any immunoglobulin naturally devoid of light chains, such that the antigen-binding capability and specificity is located exclusively in the heavy chain variable domain. Preferably, the heavy chain variable domains for use in the invention are derived from immunoglobulins naturally devoid of light chains such as may be obtained from Camelids, as described in EP-A-0584421, discussed above. The variable domain of such immunoglobulins is termed VHH (variable domain of the heavy chain of a heavy-chain antibody).

A “library” refers to a collection of nucleic acid sequences. The term “repertoire”, again meaning a collection, is used to indicate genetic diversity.

The repertoire of immunoglobulins in an organism means the totality of immunoglobulins encoded by the immune system of that organism. In the context of the present invention, which is concerned with heavy-chain antibodies (i.e. immunoglobulins naturally devoid of light chains), a repertoire of nucleic acid sequences encoding such heavy-chain antibodies, from a non-immunised source, essentially represents the complete genetic diversity of heavy-chain antibodies which can be expressed by the source organism at any given time (resulting from rearrangement of somatic DNA in cells of the immune system such as B lymphocytes). By contrast to the repertoire of immunoglobulins comprising heavy and light chains, the repertoire of heavy chain antibodies in a camelid is in the order of 10⁷ to 10⁸ and therefore the complete heavy-chain antibody repertoire of a camelid can be cloned into a suitable library. Thus a library of the present invention preferably encodes substantially the complete heavy-chain antibody repertoire of at least one source non-immunised source camelid. Accordingly, a library of the present invention, and for use in the methods of the present invention, comprises at least 10⁷, more preferably at least 2×10⁷, 5×10⁷ or 10⁸ different members.

In one embodiment, two or more libraries are obtained from two or more different donor animals and combined to produce a library having even greater diversity. Preferably libraries are pooled from 5 or more different donor animals.

Expression libraries according to the invention may be generated using conventional techniques, as described, for example, in EP-B-0368684 and EP-A-0584421. Suitably, a cDNA library comprising a plurality of nucleic acid sequences each encoding a variable domain of a heavy chain derived from an immunoglobulin naturally devoid of light chains may be generated by cloning cDNA from lymphoid cells, with or without prior PCR amplification, into a suitable expression vector.

Preferably, the nucleic acid sequences used in the method according to the invention are derived from mRNA which may suitably be isolated using known techniques from cells known to produce immunoglobulins naturally devoid of light chains. mRNA obtained in this way may be reacted with a reverse transcriptase to give the corresponding cDNA. Alternatively, the nucleic acid sequences may be derived from genomic DNA, suitably from rearranged B cells. Where genomic DNA is used, primers should be designed to amplify the rearranged immunoglobulin gene sequence, or part thereof encoding at least a region that retains the ability to exhibit antigen binding activity, and not germline sequences that have not been rearranged.

Suitable sources of heavy chain variable domains derived from immunoglobulins naturally devoid of light chains include lymphoid cells, especially peripheral blood lymphocytes, e.g. B lymphocytes, bone marrow cells, spleen cells derived from camelids.

The nucleic acid sequences encoding the heavy chain variable domains for use according to the invention are cloned into an appropriate expression vector which allows fusion with a surface protein. Suitable vectors which may be used are well known in the art and include any DNA molecule, capable of replication in a host organism, into which the nucleic acid sequence can be inserted. Examples include phage vectors (for example, lambda, T4), more particularly filamentous bacteriophage vectors such as M13. Alternatively, the cloning may be performed into plasmids, such as plasmids coding for bacterial membrane proteins or eukaryotic virus vectors.

The host may be prokaryotic or eukaryotic but is preferably bacterial, particularly E. coli.

The cloned nucleic acid sequences can be introduced into an expression vector containing nucleic acid sequences encoding one or more constant domains, such that heavy chain immunoglobulin chains may be expressed.

Preferably, the cloned nucleic acid sequences may be inserted in an expression vector for expression as a fusion protein.

The expression library according to the invention may be screened for antigen binding activity using conventional techniques well known in the art as described, for example, in Hoogenboom, Tibtech, 1997 (15), 62-70. By way of illustration, bacteriophage displaying a repertoire of nucleic acid sequences according to the invention on the surface of the phage may be screened against different antigens by a ‘panning’ process (see McCatterty, Nature, 348, (1990), 552-554) whereby the heavy chain variable domains are screened for binding to immobilised antigen. Binding phage are retained, eluted and amplified in bacteria. The panning cycle is repeated until enrichment of phage or antigen is observed and individual phage clones are then assayed for binding to the panning antigen and to uncoated polystyrene by phage ELISA.

The nucleic acid sequence encoding the antigen binding region of the heavy-chain antibody can be recovered from the phage, or other vector, by a suitable cloning process. Optionally, the sequence encoding the antigen binding region of the heavy-chain antibody can then be operably linked to other heavy chain sequences, for example to produce a complete heavy-chain antibody with the new desired specificity. The term “operably linked” means that the components described are in a relationship permitting them to function in their intended manner. Thus the sequence encoding the antigen binding region is linked to a sequence or sequences encoding other heavy chain sequences, in frame such that a functional protein can be produced in a suitable host cell.

Suitable antigens include RR-6 and di-carboxylic linoleic acid.

Preferably, the antibody fragments identified by the screening method of the present inventions have a binding affinity (Kd) for the target antigen of less than 1 μM, preferably less than 500 or 200 nM, more preferably equal to or less than 100 nM.

In one embodiment, the library of the invention is used to screen a plurality of different target antigens.

In accordance with a particular embodiment of the invention, the genes encoding the variable domains of the single domain antibodies of six individual Llamas (which had not been in contact with any of the later used antigens) were isolated and cloned into the phage display vector pHEN which allows the expression of active antibody fragments on the tip of the phage. Eleven libraries (six ‘long hinge’ and five ‘short hinge’), each containing about 10⁶ individual members were constructed, together yielding a single ‘one-pot’ library of approximately 10⁷ members with a very high level of complexity.

The library was screened for binding to RR-6 and Di-carboxylic linoleic acid using a panning process. After four and five rounds of panning a significant enrichment was observed for both antigens. After screening individual clones for specific binding activity to its antigen a large number of positive clones were identified via ELISA. Using ELISA technique the clones were shown to be highly active and exhibited strong antigen specific recognition.

In another exemplified embodiment, libraries were cloned from camel blood samples enriched for lymphocytes and also camel spleen and lymph tissue.

The resulting libraries contained about 5×10⁹ individual members. The library as screened with the following antigens: human salivary amylase, human chorionic gonadotrophin, Arthromyces ramosus peroxidase, constant domain of IgG (Fc) and Pseudomonas species. High affinity, high specificity antibodies were obtained. For example, antibodies to human chorionic gonadotrophin and Arthromyces ramosus peroxidase were shown by BlACore analysis to have affinities in the range of from 10 to 100 nM.

The following examples are provided by way of illustration only. Techniques used for the manipulation and analysis of nucleic acid materials were performed as described in Sambrook et al, Molecular Cloning, Cold Spring Harbour Press, New York, 2nd Ed. (1989), unless otherwise indicated.

HC-V denotes heavy chain variable domain.

### EXAMPLES

### Example 1

### Construction of the Naive HC-V Library

1.1 Isolation of Gene Fragments Encoding Llama HC-V Domains

A blood sample of about 200 ml was taken from an non-immunised Llama and an enriched lymphocyte population was obtained via Ficoll (Pharmacia) discontinuous gradient centrifugation. From these cells, total RNA was isolated by acid guanidium thiocyanate extraction (e.g. via the method described by Chomczynnski and Sacchi, (Anal. Biochem, 162, 156-159 (1987). After first strand cDNA synthesis (e.g. with the Amersham first strand cDNA kit), DNA fragments encoding HC-V fragments and part of the long or short hinge region where amplified by PCR using specific primers:

| | | |
|---|---|---|
| | PstI | |
| VH-2B | 5′-AGGTSMARCTGCAGSAGTCWGG-3′. | (see SEQ. |
| | | ID. NO: 1) |
| | SfiI | |
| PCR.162: | 5′-CATGCCATGACTCGCGGCCCAGCCGG | (see SEQ. |
| | CCATGGCCSAGGTSMARCTGCAGSAGTCW | ID. NO: 2) |
| | GG-3. | |
| | S = C and G, M = A and C, | |
| | R = A and G ,W = A and T, | |
| | HindIII NotI | |
| Lam-07: | 5′-AACAGTTAAGCTTCCGCTTGCGGCCG | (see SEQ. |
| | CGGAGCTGGGGTCTTCGCTGTGGTGCG-3′. | ID. NO: 3) |
| | HindIII NotI | |
| Lam-08: | 5′-AACAGTTAAGCTTCCGCTTGCGGCCG | (see SEQ. |
| | CTGGTTGTGGTTTTGGTGTCTTGGGTT-3′. | ID. NO: 4) |

Upon digestion of the PCR fragments with PstI (coinciding with codon 4 and 5 of the HC-V domain, encoding the amino acids L-Q) and NotI (located at the 3′-end of the HC-V gene fragments), the DNA fragments with a length between 300 and 400 bp (encoding the HC-V domain, but lacking the first three and the last three codons) were purified via gel electrophoresis and isolation from the agarose gel. NotI has a recognition-site of 8 nucleotides and it is therefore not likely that this recognition-site is present in many of the created PCR fragments. However, PstI has a recognition-site of only 6 nucleotides. Theoretically this recognition-site could have been present in 10% of the created PCR fragments, and if this sequence is conserved in a certain class of antibody fragments, this group would not be represented in the library cloned as PstI-NotI fragments. Therefore, a second series of PCR was performed, in which the primary PCR product was used as a template (10 ng/reaction). In this reaction the 5′ VH2B primer was replaced by PCR162. This primer introduces a SfiI recognition-site (8 nucleotides) at the 5′ end of the amplified fragments for cloning. Thus, a total of 24 different PCR products were obtained, four (short and long hinge, Pst I/Not I and Sfi I/Not I) from each Llama. Upon digestion of the PCR fragments with SfiI (upstream of the HC-V coding sequence, in the pelB leader sequence) and NotI, the DNA fragments with a length between 300 and 400 bp (encoding the HC-V domain) were purified via gel electrophoresis and isolation from the agarose gel.

1.2 Construction of HCV Library in pHEN.5

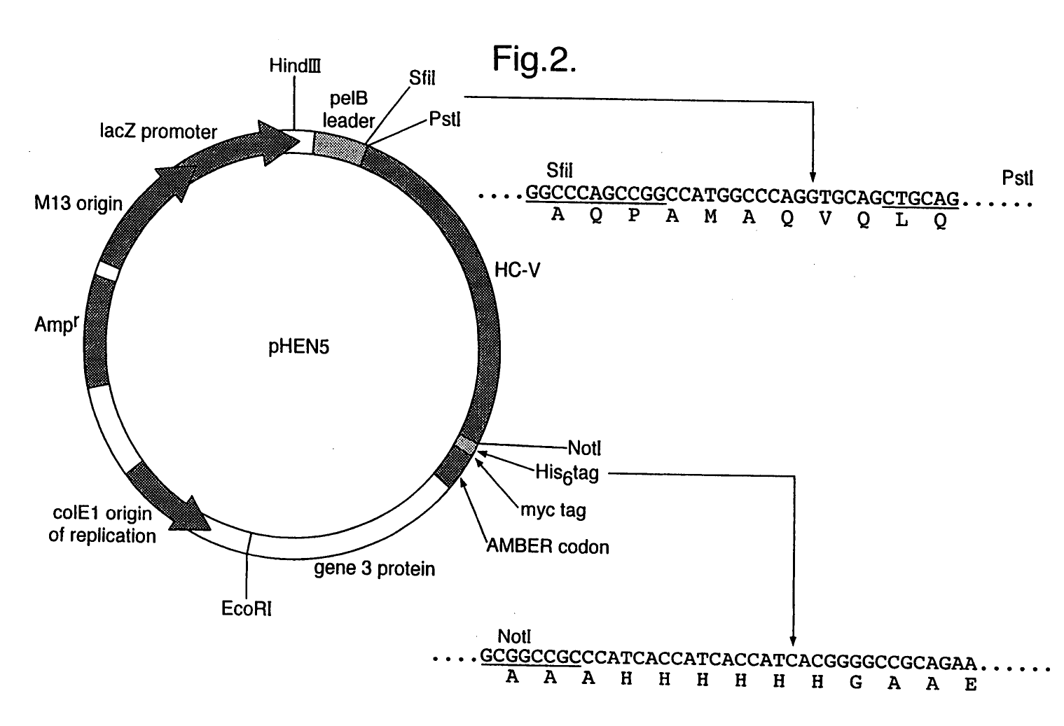

The Pst I/Not I or Sfi I/Not I—digested fragments were purified from agarose and inserted into the appropriately digested pHEN.5 vector (FIG. 2). Prior to transformation, the ligation reactions were purified by extraction with equal volumes of phenol/chloroform, followed by extraction with chloroform only. The DNA was precipitated by addition of 0.1 volume 3M NaAc pH5.2 and 3 volumes ethanol. The DNA pellets were washed ×2 with 1 ml 70% ethanol, dried and resuspended in 10 μl sterile milliQ water. Aliquots were transformed into electrocompetent Ecoli XL1-Blue (Stratagene) by electroporation, using a Bio-Rad Gene Pulser. The protocol used was as recommended by Stratagene. The final library, consisting of approximately 7.8×10⁶ individual clones, was harvested by scraping the colonies into 2TY+Ampicillin (100 ug/ml)+Glucose (2% w/v) culture medium (35-50 ml each). Glycerol stocks (30% v/v) and DNA stocks were prepared from these and stored at −80° C.

### Example 2

### Selection of HC-V Fragments which Exhibit Antigen Binding Affinity

2.1 Panning of the Library

Two ‘antigens’ were used for screening the naive phage-displayed HCV library; Di acid-OVA (dicarboxylic linoleic acid-ovalbumin conjugate) and the azo-dye RR6 (available from ICI) conjugated to BSA (reactive red six-bovine serum albumin conjugate).

Phages displaying antibody fragments on their surface were obtained using the following protocol:

Phage rescue:

15 mL 2TY/Ampicillin/Glucose was incubated with 100 μL of a glycerol stock of the naive library culture. The culture was allowed to grow until log-phase (A₆₀₀=0.3-0.5), at which point 4.5×10⁹ pfu M13K07 helper phage were added. After infection for 30 minutes at 37° C. (without shaking) the infected cells were spun down (5000 rpm for 10 minutes) and the pellet was resuspended in 200 mL 2×TY/Ampicillin/Kan. After incubation with shaking at 37° C. overnight, the culture was spun and the paheges present in the supernatant were precipitated by adding ⅕ volume PEG/NaCL (20% Polyethylene glycol 8000, 2.5M NaCL).

After incubation on ice-water for 1 hour the phage particles were pelleted by centrifugation at 8000 rpm for 30 minutes. The phage pellet was resuspended in 20 mL water and re-precipitated by adding 4 mL PEG/NaCl solution. After incubation in ice-water for 15 minutes the phage particles were pelleted by centrifugation at 5000 rpm for 15 minutes and resuspended in 2 mL PBST with 2% Marvel (milk powder; trade name)(plus 2% OVA for the Di acid-OVA tube and 2% BSA for the RR6-BSA tube).

Panning:

The PEG precipitated phages in PBST/2% Marvel (0.5 ml) (plus 2% OVA for the Di acid-OVA tube and 2% BSA for the RR6-BSA tube) were added to Nunc-immunotubes (5 mL) coated with 1 ml Di acid-OVA conjugate (100 μg/ml), 1 ml RR6-BSA conjugate (100 μg/ml) or a control tube. All tubes were blocked with PBST/2% Marvel) (plus 2% OVA for the Di acid-OVA tube and 2% BSA for the RR6-BSA tube) at 37° C. for 1 hour before the phages were added. After incubation for 3-4 hours at room temperature, unbound phage were removed by washing the tube 20 times with PBS-T followed by 20 washes with PBS. The bound phages were eluted by adding 1 mL elution buffer (0.1M HCL/glycine pH2.2/1 mg/mL BSA). The elution mixture was neutralised with 60 μL 2M Tris, and the eluted phages were added to 9 mL log-phase E. coli XL-1 Blue. Also 4 mL log-phase E. coli XL-1 Blue were added to the immunotube. After incubation at 37° C. for 30 minutes to allow infection, the 10 mL and 4 mL infected XL-1 Blue bacteria were pooled and plated onto SOBAG plates (20 g bacto-tryptone, 5 g bacto-yeast extract, 0.1 g Na C1, 15 g Agar; made up to 1 litre with distilled water and autoclaved, allowed to cool and 10 mL MgC1₂ and 27.8 mL 2M glucose added. Following growth overnight at 37° C. the clones obtained from the antigen sensitised tubes were harvested and used as starting material for the next round of panning, or alternatively individual colonies were assayed specific antigen binding activity.

For panning rounds 1 to 3 there was no indication of phage enrichment over background for both antigens (Table 1). However, at pan 4, significant enrichment of phages was observed for both RR6-BSA and Di-acid-OVA.

TABLE 1
Results of the panning reactions (fold enrichment over
background)

| | Panning Antigen | | | | |
|---|---|---|---|---|---|
| | Pan 1 | Pan 2 | Pan 3 | Pan 4 | Pan 5 |
| RR6 | none | none | none | 100-fold | ˜200-fold |
| Di-acid | none | none | none | ˜100-fold | 50-100-fold |

### Example 3

### Identification of Individual HC-V Fragments with Antigen Binding Activity

Individual bacterial colonies were picked (200 from pans 4 and 5, for both antigens) using sterile toothpicks and added to the wells of 96-well microtitre plates (Sterilin) each containing 100 ml of 2TY, 1% (w/v) glucose and ampicillin (100 mg/ml). After allowing the cultures to grow overnight at 37° C., 20 μl aliquots from each well of these ‘masterplates’ were added to the wells of fresh microtitre plates each containing 200 ml of 2TY, 1% glucose, 100 mg/ml ampicillin, 10⁹ M13KO7 helper phage. Infection at 37° C. for 2.5 h was followed by pelleting the cells and resuspending the infected cells in 200 ml of 2TY containing ampicillin (100 mg/ml) and kanamycin (25 mg/ml). Following overnight incubation at 37° C., the phage-containing supernatants (100 μl) were added to the wells of Sterilin microtitre plates containing 100 μl/well of the appropriate blocking buffer (same buffer used as during panning reactions). Pre-blocking of the phage was carried out in these plates for 30 mins at room temp. After 30 minutes at room temperature, 100 μl of phage supernatant was added to the wells of a Greiner HC ELISA plate coated with the corresponding antigen, and to the wells of an uncoated plate. After 2 h incubation at 37° C. unbound phages were removed, and bound phages were detected with rabbit anti-M13 followed a goat anti-rabbit alkaline phosphatase conjugate. The assays were developed with 100 ml/well of p-nitrophenyl phosphate (1 mg/ml) in 1M diethanolamine, 1 mM MgCl₂, pH9.6 and the plates read after 5-10 mins at 410 nm.

TABLE 2
Percentage of panned phage clones which specifically
recognise and bind immobilised antigen.

| Panning Antigens | Pan 4 | Pan 5 |
|---|---|---|
| RR6-BSA | 23% | 43% |
| Diacid-OVA | 13% | 20% |

### Example 4

### Characterisation of HC-V Fragments with Specific RR-6 Binding Activity

To test the individual clones identified in the phage ELISAs for their ability to produce active soluble antibody fragments, plasmid DNA from 12 clones that were shown to specifically recognise RR6-BSA was isolated and used to transform the non-suppressor E. coli strain D29AI. Commercially available strains such as TOPIOF (stratagene) and HB2151 (Pharmacia) may alternatively be used. Two transformants of each clone were pre-grown in 10 ml 2TY/Ampicillin/Glucose. After 3-4 hours of growth at 37° C. (OD₆₀₀=0.5), the cells were pelleted by centrifugation and resuspended in 5 ml 2TY/Ampicillin/IPTG (0.1 mM). After 24 hours of incubation at 25° C. the cultures were centrifuged, and the supernatants were analysed for the production of antigen binding activity in essential the same way as described in Example 3. In this case, however, the presence of specifically bound HC-V fragments was detected by incubation with monoclonal anti-myc antibodies, followed by incubation with poly-clonal rabbit-anti-mouse conjugate with alkaline phosphatase.

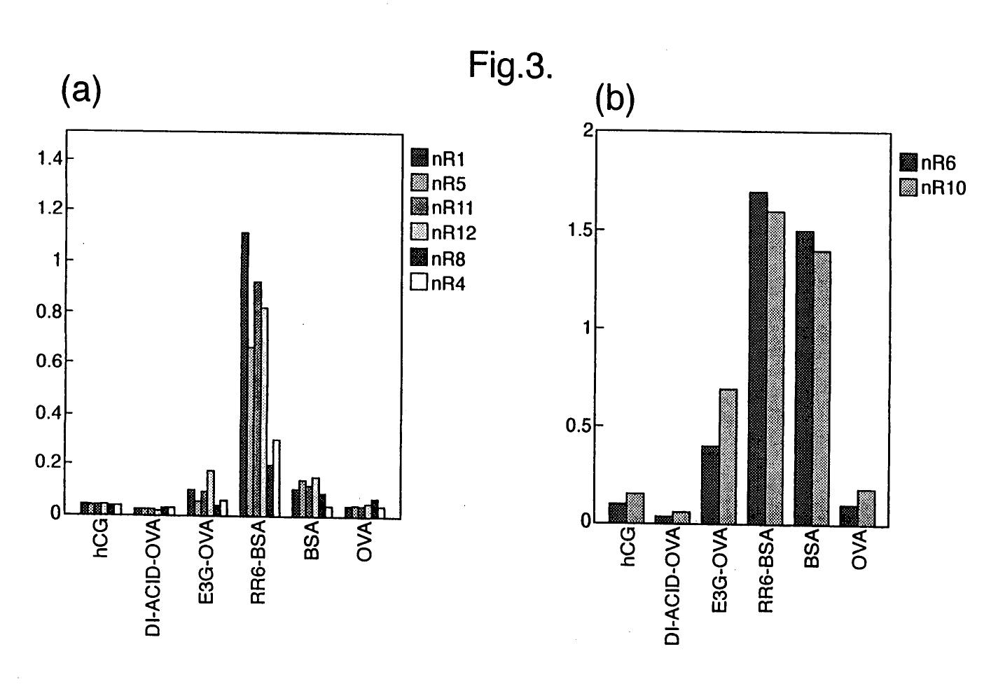

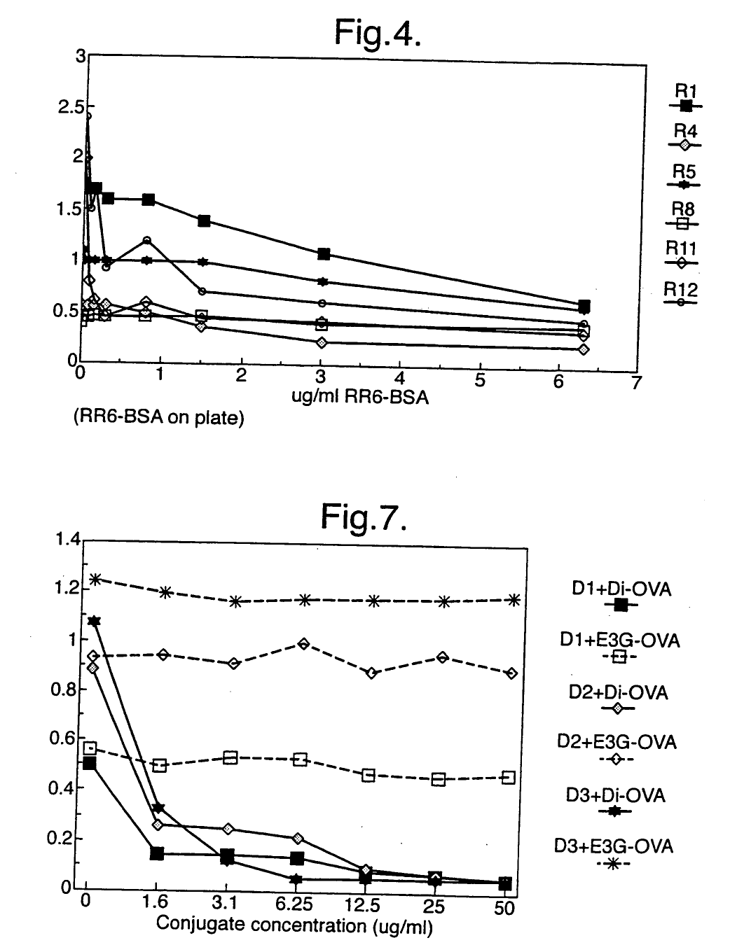

As shown in FIG. 3A, six (nR1, nR2, nR5, nR7, nR11 and nR12) out of the twelve chosen RR6-BSA—panned clones were specific for RR6-BSA, and did not bind to any of the other antigens tested. The specificity of these 6 clones was also confirmed in competition assays in which following the protocol outlined above, soluble RR6 or RR6-BSA conjugate was present during the antigen binding reaction and was shown to reduce the specific binding signal (FIG. 4). Another three clones (nR3, nR4 and nR8) were specific for RR6-BSA, but the signals observed were very low. These weak ELISA signals correlated with relatively poor signals in dot-blot experiments, indicating that these clones were poor producers of soluble fragment. This was confirmed by analysis of the supernatants on Western blots (FIG. 3B). The remaining 3 clones (nR6, nR9 and nR10) gave significant signals over background on RR6-BSA, BSA and E3G-OVA (FIG. 3A). It would appear that these three ‘sticky’ clones bind to immobilised proteins in general.

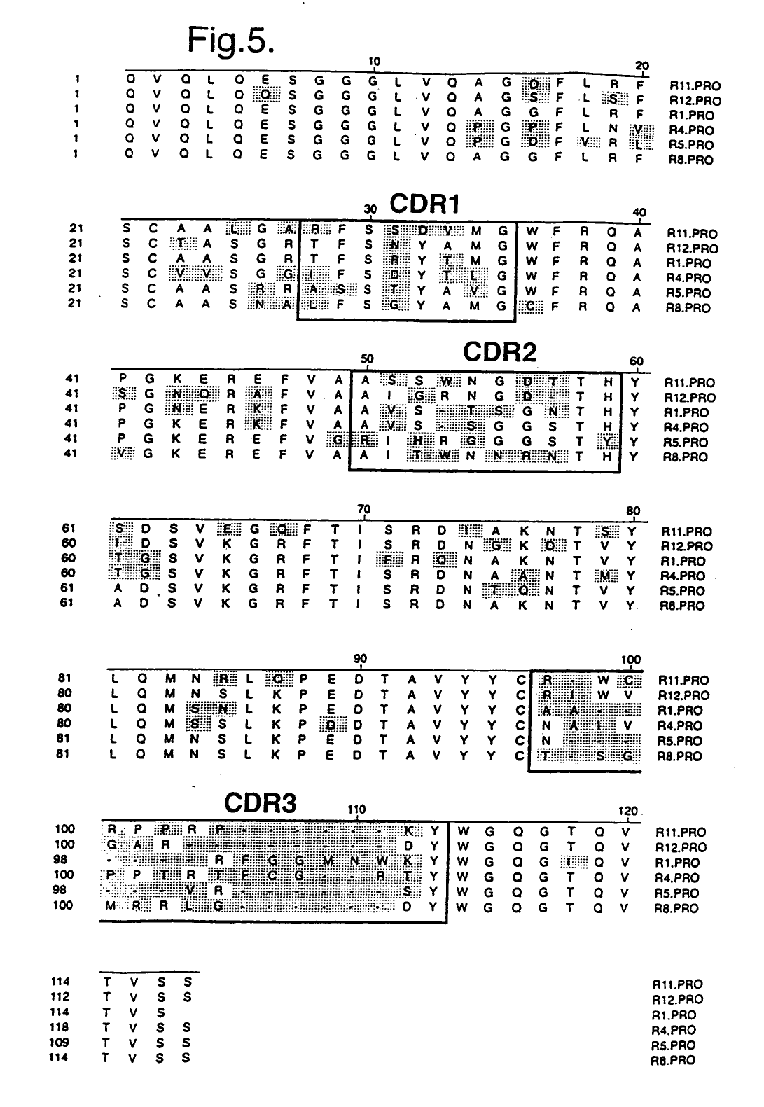

The sequence of the isolated anti-RR6 HC-V fragments are listed in FIG. 5.

| | |
|---|---|
| nR1. | (SEQ. ID. NO: 5) |
| nR4. | (SEQ. ID. NO: 6) |
| nR5. | (SEQ. ID. NO: 7) |
| nR8. | (SEQ. ID. NO: 8) |
| nR11. | (SEQ. ID. NO: 9) |
| nR12. | (SEQ. ID. NO: 10) |

### Example 5

### Characterisation of HC-V Fragments with Specific Di-Carboxylic Acid Binding Activity

To test the individual clones identified in the phage ELISAs for their ability to produce active soluble antibody fragments, plasmid DNA from 9 clones that were shown to specifically recognise Di Acid-OVA was isolated and used to transform the non-suppressor E. coli strain D29AI. Two transformants of each clone were pre-grown in 10 ml 2TY/Ampicillin/Glucose. After 3-4 hours of growth at 37° C. (OD₆₀₀=0.5), the cells were pelleted by centrifugation and resuspended in 5 ml 2TY/Ampicillin/IPTG (0.1 mM). After 24 hours of incubation at 25° C. the cultures were centrifuged, and the supernatants were analysed for the production of antigen binding activity in essential the same way as described in Example 3. In this case, however, 1% gelatin was used as the blocking reagent and the presence of specifically bound HC-V fragments was detected by incubation with monoclonal anti-myc antibodies, followed by incubation with poly-clonal rabbit-anti-mouse conjugate with alkaline phosphatase.

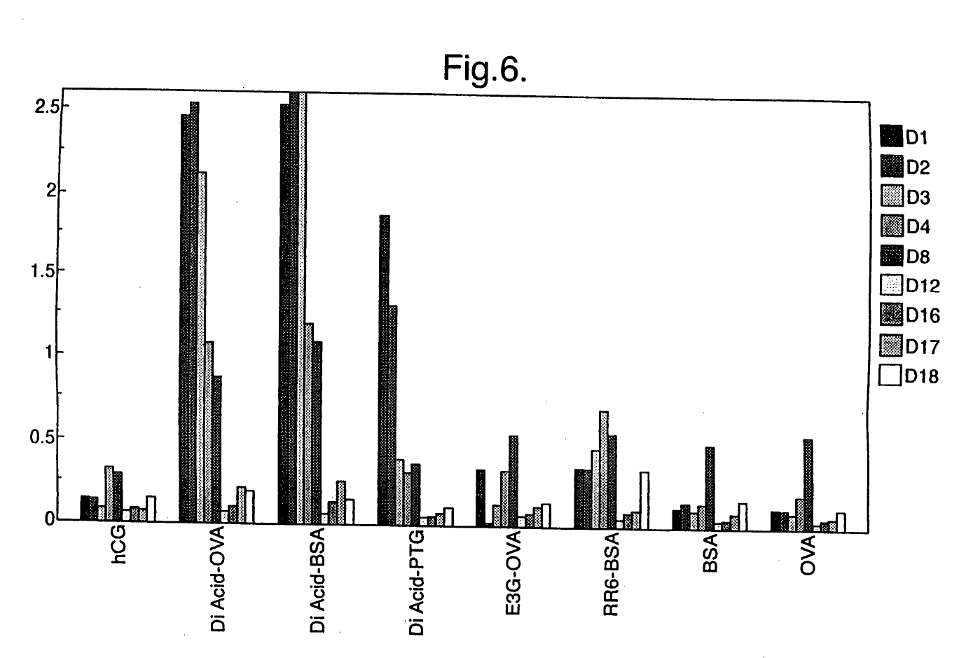

Three of the selected HC-V samples gave high signals against Di acid conjugated to OVA, BSA or PTG (porcine thyro globulin), and background signals against all other immobilised antigens tested (FIG. 6). Much lower signals for Di acid-OVA were observed for a further 2 clones (FIG. 6). The specificity of the 3 leading clones was further demonstrated using competition assays as described in Example 4, which showed strong inhibition of Di-Acid-OVA binding of these clones when supernatants were preincubated with Di acid-OVA conjugate, whereas the same concentration range of the E3G-OVA conjugate had no inhibitory effect (FIG. 7).

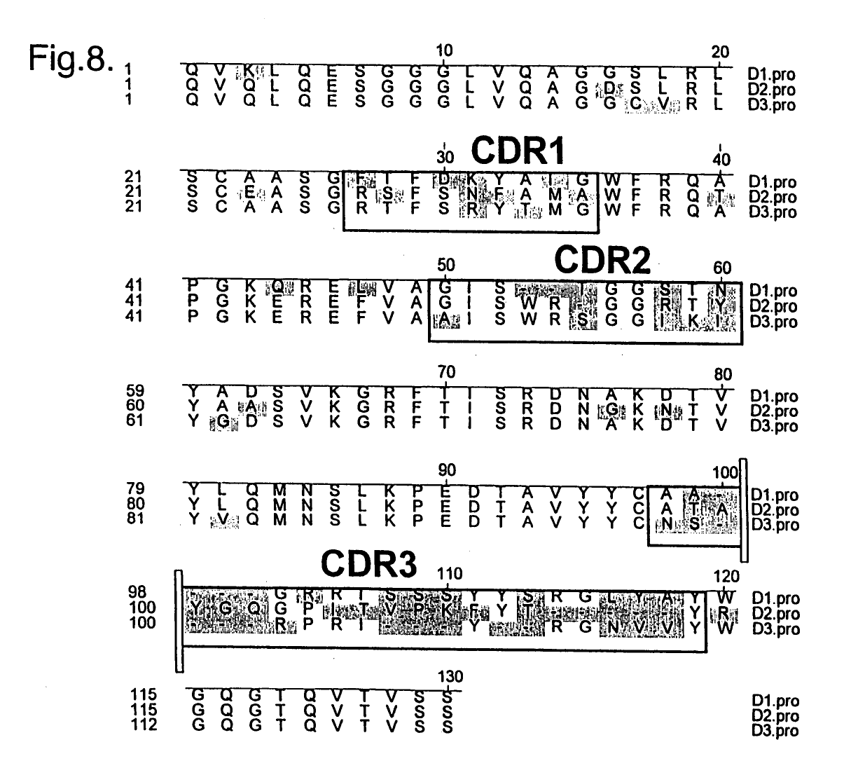

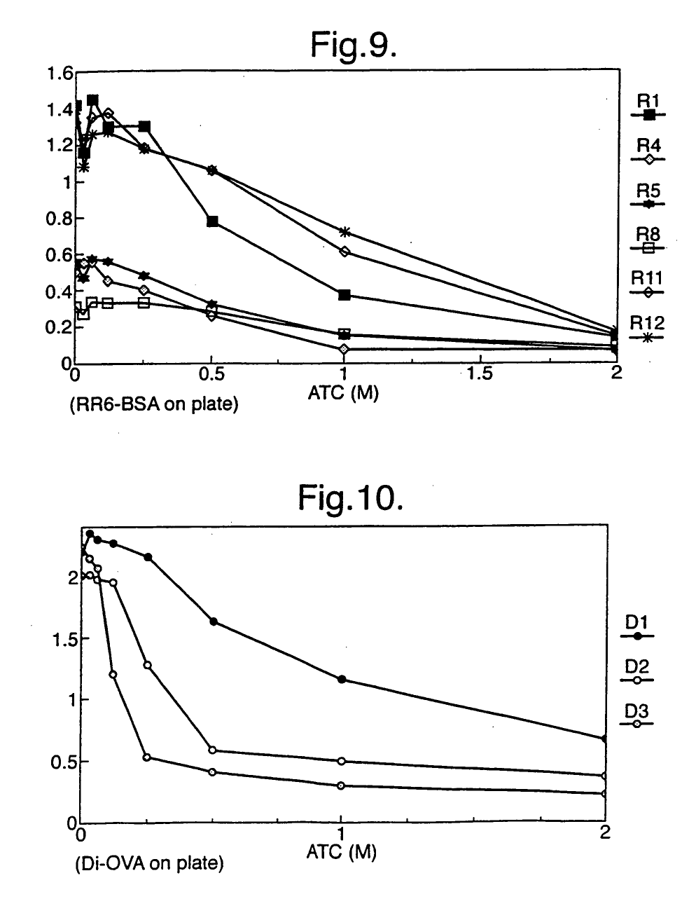

The sequence of the isolated anti-Di Acid HC-V fragments are listed in FIG. 8.

| | |
|---|---|
| nD1. | (SEQ. ID. NO: 11) |
| nD2. | (SEQ. ID. NO: 12) |
| nD3. | (SEQ. ID. NO: 13) |

### Example 6

### Construction and Screening of a Naïve Camel VHH Library

Materials and Methods

Amplification of the Naive Camel VHH Repertoires

From two naive camels, a blood sample of about 150 ml was taken and an enriched lymphocyte population was obtained via centrifugation on a Ficoll (Pharmacia) discontinuous gradient. Furthermore, from four camels 0.5 gram of spleen and lymph tissue was homogenised with a thorax (each sample containing approximately 10⁸ lymphocytes).

From each sample, total RNA was isolated by acid guanidium thiocyanate extraction (Chomczynski and Sacchi, 1987, Analytical Biochem. 162: 156-159) with minor variations. Cell pellets containing 1-5×10⁸ cells were directly resuspended in 4 ml 4 M guanidinium-SCN, 25 mM citric acid, pH 7, containing 0.5% sarkosyl and 1% v/v 2-mercapto-ethanol. This lysis buffer was freshly made with DEPC-treated water (Di-ethyl pyrocarbonate, ex Sigma). To break the viscosity, syringes of different diameters (first 0.8×50, then 0.5×16) were used to shear the chromosomal DNA after which the RNA was isolated by phenol extraction. The phenol extraction was performed by adding 4 ml phenol (saturated with DEPC-water) and 400 μl 2 M NaAc pH 4.0. After vigorous mixing, 2 ml chloroform/isoamylalcohol (24:1) (CIAA) was added, mixed and kept on ice for 15 min. After centrifugation for 10 minutes at 3,000 g the water phase was transferred to a clean Falcon tube and extracted with phenol and CIAA again. A first ethanol precipitation was performed by adding 0.75 volume 100% ethanol and incubating overnight at −200 C. The RNA was collected by centrifugation (HB4, 16,300 g, 20 minutes) and the pellet was resuspended in 400 μl of DEPC-water. A second ethanol precipitation was performed by adding 2.5 volumes of 100% ethanol and 0.1 volume 2 M NaAc pH4.0 (OPBIC 227/01).

After centrifugation at 15,300 g (30 minutes) the supernatant was removed, dried in a speedvac and the pellet is resuspended in 200 μl DEPC-water. The total amount of RNA was determined by measuring the OD₂₆₀ (for RNA an OD of 1 corresponds with 40 μg/ml). Messenger RNA was isolated from total RNA by using the Oligotex mRNA mini kit (Qiagen, no 70022) according to the suppliers protocol (OPBIC 226-3).

Subsequently, first strand cDNA was synthesized using the Amersham first strand cDNA kit (RPN1266). In a 20 μl reaction mix 0.4-1 μg mRNA was used. The poly-T primer was used to prime the first DNA strand. After cDNA synthesis, the reaction mix was directly used for amplification by PCR.

DNA fragments encoding VHH antibody fragments were amplified via two separate routes

Route A:

In route A, VHH encoding gene fragments were amplified in a single PCR reactions (Perkin Elmer DNA Thermal Cycler 480). From the total of 60 μl of cDNA template that was made, 10 μl cDNA was used in 10 separate PCR reactions of 50 μl. PCR reactions were performed with Amplitaq gold as described by manufacturer. Primers were applied in 100 pM concentrations and the PCR reaction was performed as follows: 1 cycle 12′ 94° C.; 28 cycles 30″ 94° C., 1′ 55° C., 2′ 72° C.; 1 cycle 5′ 72° C. In this reaction the 5′ end of the framework 1 region and the upstream part of the short or long hinge region were used were used to amplify VHH specific gene fragments.

Route B

In route B, VHH encoding gene fragments were amplified by making use of two separate PCR reactions independent of the hinge region. The primary PCR was performed as described above, but with newly designed primers in Framework 1 region and in the constant domain CH2. Five PCR reactions of 50 μl were performed per camel for each mix (1 μl cDNA template per reaction). All 16 PCR fragments were separated on 1.5% agarose gels and DNA fragments between 470 and 590 base pairs were isolated by means of the Qiaex-II extraction kit (30 μl glass milk per fragment). Subsequently, the isolated DNA fragments were used as templates in a secondary PCR reaction. On each template two PCR reactions were performed. Primers were used for 5′ priming onto framework 1 region sequences and introduction of the SfiI restriction site and for 3′ priming onto framework 4 sequences. Four PCR reactions of 50 μl were performed per template from the primary PCR and amplificates were obtained by 20 PCR cycles instead of 28.

Purification and Digestion of PCR Fragments.

DNA fragments obtained via route A were pooled per camel and per short or long hinge VHH type. Furthermore, the fragments derived from blood, lymph and spleen were kept separate. Corresponding tubes from 20 independent VHH fragment repertoires were pooled (total 250 μl/fragment pool) and separated on 1.5% agarose gels. DNA fragments with a length between 300 and 400 base pairs were isolated by means of the Qiaex-II extraction kit (150 μl glass milk per fragment). The purified DNA-fragments were digested with PstI (coinciding with codon 4 and 5 of the VHH domain, encoding the amino acids L-Q) and NotI (directly C-terminal of the VHH sequence). Subsequently, the digested PCR-products were purified with Qiaquick PCR purification columns from Qiaex according to supplier.

DNA fragments obtained via route B were pooled per camel and per mix 1 or 2. As in route A, fragments derived from blood, lymph and spleen were kept separate. The corresponding tubes from 20 independent VHH fragment repertoires were pooled (total 200 μl/fragment pool) and separated on agarose gels and purified as described above. The purified DNA-fragments were digested with SfiI and NotI and purified as described above.

Cloning of VHH Repertoires in Phage Display Vector.

For the construction of this naive camel antibody fragment library, the antibody fragment repertoires from route A were cloned into a suitable phage display vector by digesting both with PstI and NotI and the fragments obtained via route B were cloned into the vector by SfiI/NotI digestions. Ligations were performed with ligation buffer and ligase from Promega according to the instructions of the manufacturer. After the overnight ligation at room temperature, ligation mixes were desalted by spin dialysis on microcon YM-30 centrifugal filters. The ligation mixes were dialysed by three changes with sterilised deionised water.

Creation of VHH Libraries.

The end volume of the ligation mixes was approximately 80 μl for route A and 50 μl for route B. Three batches of 20 μl mix of route A and three batches of 20 μl mix of route B were transformed into electro competent E. coli TG1 cells (see OPGTF 1803). Per transformation 100 μl cells was mixed with ligation mix and transferred in Bio-Rad electro-cuvettes (0.2 mm gap version). The Bio-Rad Gene Pulser was set at 2.5 kV, 200% and 25 μF. Typical time constants were 4.8 ms. After transformation, 1.5 ml of fresh 2TY medium was added to each cuvette and cells were regenerated for one hour at 37° C. Subsequently, corresponding transformations were pooled and plated onto 2TY agar plates containing glucose and ampicillin. After overnight growth at 37° C., plates containing the transformants were scraped and the cells collected in 2TY glu/amp medium. For storage at −80° C., glycerol was added to a final concentration of 20% (v/v).

Full-Length and Fingerprint Analysis.

To check the quality the constructed antibody fragment libraries, individual clones containing a VHH expression construct were tested for the presence of a full-length VHH encoding insert sequence. Furthermore, the diversity of the tested set of clones was analysed by performing Hinfl fingerprint analysis. The full length and the Hinfl digested PCR products were analysed on 2% agarose gels.

Large Scale Helper Phase Production.

A phage plaque (VCSM13) was inoculated into 34 ml 1/100 diluted log phase E. coli TG1 and grown for about 2 hrs at 37° C. without shaking. Subsequently, this culture is diluted into 100 ml 2TY and grown for 1 hr at 37° C. with shaking in a 2 litre baffled shake flask. Then, kanamycin was added to a final concentration of 50 lg/ml and grown overnight at 37° C. with shaking. After this phage production phase, the culture was centrifuged at 4000 g for 15 min. The supernatant was then added to ¼ volume of 20% PEG 6000, 2.5 M NaCl and incubated on ice for 30-45 min. Subsequently, phages were isolated by centrifugation at 4,000 g for 20 min. The resulting phage pellet was resuspended in 5 ml sterile PBS and passed through a 0.45 lm filter. Finally, the phages were diluted in PBS to make a stock solution of approximately 1×10¹² pfu/ml.

Selection of Specific Antibody Fragments From the Library.

For production of phage sub-libraries derived from lymph, spleen and blood were kept separate. Of the route-A sub-libraries, 7 sub-libraries derived from lymph, 22 derived from spleen and 4 derived from blood were inoculated in 2TY-glu/Amp.

For the route-B sub-libraries we inoculated 6 sub-libraries derived from lymph, 12 derived from spleen and 3 derived from blood (10⁸ TG1 transformants per sub-library were inoculated, see appendix C). The A and the B library were kept separate and for the blood and the lymph derived sub-libraries 250 μl of each mix was inoculated in 250 ml 2TY-glu/Amp. At OD600=0.7, 100 ml of the culture was infected with 4.5×10¹¹ pfu of VCSM13 helper phage. Subsequently, infected cells collected by centrifugation (4000 g, 10′) were resuspended in 800 ml 2TY-Amp/Kan. For the spleen derived sub-libraries all volumes were multiplied by 4. Phages were then rescued according to Marks et al., 1991, Journal of molecular biology 222: 581-597. For selections approximately 1013 cfus were used per selection with antigens immobilised in immunotubes (biopanning) or with soluble biotinylated antigens. The amount of antigen coated in immunotubes (Maxisorp) was 100 μg/ml during round 1, 35 μg/ml in round 2 and 12.5 μg/ml in round 3. For the soluble selections 100 nM of biotinylated antigen was used in round 1, 35 nM in round 2 and 12.5 nM in round 3 unless stated otherwise. Antigens were biotinylated at a ratio of 10 to 20 molecules of NHS-EZlinked-Biotin (Pierce) per molecule antigen according to suppliers recommendations. Efficiency of biotinylation was checked in ELISA by incubating hSA-biotin and hCG-biotin (both 1 μg/ml) in a streptavidin (5 μg/ml) coated Maxisorp plate followed by the addition the anti-hSA VHH fragment 2B5 and the anti-hCG VHH H14, respectively. After adding anti-VHH serum R906 (1:4,000) and swine-anti-rabbit-IgG-HRP (1:5,000) binding was visualised as described in OPGTF1506-2. ARP-biotin was added to a streptavidin coated maxisorp plate (1 μg/ml) followed by the addition of streptavidin-HRP conjugate (1:1,000). All biotin conjugates gave specific signals indicating an efficient reaction for each antigen (data not shown). The selections with ARP were also performed by panning in maxisorp immuno-tubes. For this type of selection no biotinylation of the antigen was necessary.

Screening of Selected Clones.

Individual E. coli TG1 clones from each round of selection for every tested antigen were grown in microtiter plates and the production of VHH fragments was induced by addition of IPTG (0.1 mM). Culture supernatants containing free VHH domains were tested in ELISA for binding to their specific antigen by using the primary 9E10 anti-myc (1:2,000) antibody or the polyclonal anti-llama antibody serum (1:5,000) and secondary anti-mouse HRP conjugate, anti-rabbit HRP (Dako, 1:3,000) or for ARP anti-mouse AP conjugate (Promega, 1:5,000) for detection, respectively (see OPGTF1506-2).

Results

Library Construction and Quality Control.

From the blood of two camels and the lymph and spleen tissue of four camels, RNA from the isolated B-lymphocytes was transcribed into cDNA, which was used as a template in an amplification reaction either via route A or route B. In route A, antibody fragment encoding DNA fragments were amplified in a single PCR reaction using the introduced PstI and NotI restriction sites for cloning into the phage display vector pUR8102. In route B these fragments were amplified in two subsequent PCR reactions independent of the hinge region. DNA fragments obtained via this strategy were cloned into pUR8102 after SfiI/NotI digestion. Ligation mixes were transformed into electrocompetent E. coli TG1 cells and transformed cells were grown on selective 2TY agar plates. Transformants were collected from the plates and stored as glycerol stocks (for details see Materials and Methods). In table 3, the sizes of all sub-libraries and the OD600 of the glycerol stocks are presented. The final naive camel VHH library has a size of 5.2×10⁹.

TABLE 3
Sizes of naive camel sub-libraries obtained via amplification routes
A and B.

| Camel | Route A | | | | Route B | | | |
|---|---|---|---|---|---|---|---|---|
| Sub- | Short hinge | | Long hinge | | Mix 1 | | Mix 2 | |
| library^{a} | Size^{b} | OD600^{c} | Size | OD600 | Size | OD600 | Size | OD600 |
| L1 | 4.5 × 10⁶ | 52 | 1.7 × 10⁸ | 55 | 1.1 × 10⁸ | 62 | 4.5 × 10⁷ | 51 |
| L2 | 1.1 × 10⁷ | 49 | 4.5 × 10⁷ | 53 | 8.0 × 10⁷ | 55 | 8.5 × 10⁷ | 53 |
| L3 | 5.0 × 10⁶ | 40 | 3.9 × 10⁷ | 51 | 6.4 × 10⁷ | 56 | 7.0 × 10⁷ | 60 |
| L5 | 3.5 × 10⁷ | 57 | 4.5 × 10⁷ | 59 | 4.8 × 10⁸ | 58 | 4.0 × 10⁷ | 58 |
| S1.1^{d} | 1.0 × 10⁸ | 62 | 1.4 × 10⁸ | 58 | 1.8 × 10⁸ | 60 | 1.8 × 10⁸ | 59 |
| S1.2 | 5.0 × 10⁷ | 50 | 8.6 × 10⁷ | 62 | 5.4 × 10⁷ | 62 | 1.1 × 10⁸ | 52 |
| S1.3 | 1.6 × 10⁸ | 55 | 6.5 × 10⁸ | 55 | | | | |
| S2.1 | 4.7 × 10⁷ | 53 | 8.2 × 10⁷ | 52 | 9.0 × 10⁷ | 57 | 6.0 × 10⁷ | 58 |
| S2.2 | 8.4 × 10⁷ | 56 | 2.7 × 10⁷ | 50 | 7.2 × 10⁷ | 49 | 3.5 × 10⁷ | 50 |
| S2.3 | 1.8 × 10⁸ | 49 | 9.0 × 10⁷ | 54 | | | | |
| S3.1 | 7.8 × 10⁷ | 53 | 1.0 × 10⁸ | 60 | 18 × 10⁸ | 58 | 9.0 × 10⁷ | 57 |
| S3.2 | 1.0 × 10⁸ | 54 | 1.2 × 10⁸ | 54 | 9.0 × 10⁷ | 47 | 6.3 × 10⁷ | 52 |
| S3.3 | 1.2 × 10⁸ | 50 | 1.5 × 10⁸ | 51 | | | | |
| S8.1 | 5.4 × 10⁷ | 59 | 1.0 × 10⁸ | 55 | 9.0 × 10⁷ | 58 | 1.1 × 10⁸ | 60 |
| S8.2 | 4.9 × 10⁷ | 51 | 4.8 × 10⁷ | 50 | 9.7 × 10⁷ | 48 | 9.0 × 10⁷ | 53 |
| S8.3 | 6.0 × 10⁷ | 52 | 6.0 × 10⁷ | 52 | | | | |
| B1 | 1.2 × 10⁸ | 56 | 8.6 × 10⁷ | 62 | 9.0 × 10⁷ | 56 | 9.0 × 10⁷ | 50 |
| B4 | 3.5 × 10⁷ | 48 | 4.2 × 10⁷ | 52 | 6.3 × 10⁷ | 49 | 7.2 × 10⁷ | 50 |

^{a}L = derived from lymph; S = derived from spleen; B = derived from blood.
^{b}Total size of sub-libraries as number of transformed TG1 cells
^{c}OD660 of glycerol stock stored in −80° C.
^{d}From each spleen tissues derived antibody fragment repertoires several libraries were created

To evaluate the quality of the different sub-libraries, 20 clones of each library were analysed for the presence of a full-length VHH insert and for diversity. Inserts were amplified from whole cells by making use of colony PCR. Subsequently, these DNA fragments were digested with the HinfI restriction enzyme which frequently cuts within VHH gene segments. This analysis showed that cloning was very efficient and that diversity of VHH gene fragments was high for almost each sub-library (table 4).

TABLE 4
Quality control of constructed libraries.

| | Route A | | | | Route B | | | |
|---|---|---|---|---|---|---|---|---|
| Camel | Short hinge | | Long hinge | | Mix 1 | | Mix 2 | |
| sub | | div. | | div. | | div. | | div. |
| library^{a} | % FL^{b} | f.p.^{c} | % FL | f.p. | % FL | f.p. | % FL | f.p. |
| L1 | 95 | 13 | 95 | 13 | 55* | 8 | 75 | 10 |
| L2 | 85 | 12 | 90 | 14 | 95 | 7 | 95 | 10 |
| L3 | 40* | 5 | 95 | 9 | 65* | 8 | 100 | 9 |
| L5 | 95 | 11 | 100 | 13 | 75 | 8 | 95 | 9 |
| S1.1 | 60* | 8 | 75 | 9 | 90 | 4 | 90 | 6 |
| S1.2 | 55* | 8 | 95 | 12 | 100 | 6 | 100 | 8 |
| S1.3 | 75 | 9 | 100 | 14 | | | | |
| S2.1 | 90 | 11 | 90 | 12 | 60* | 6 | 55* | 4 |
| S2.2 | 95 | 11 | 85 | 12 | 60* | 8 | 55* | 5 |
| S2.3 | 85 | 12 | 100 | 9 | | | | |
| S3.1 | 100 | 11 | 80 | 7 | 95 | 11 | 95 | 11 |
| S3.2 | 95 | 12 | 85 | 13 | 90 | 10 | 90 | 11 |
| S3.3 | 90 | 9 | 95 | 11 | | | | |
| S8.1 | 90 | 12 | 100 | 12 | 90 | 11 | 100 | 9 |
| S8.2 | 90 | 8 | 95 | 8 | 85 | 10 | 100 | 11 |
| S8.3 | 95 | 9 | 100 | 7 | | | | |
| B1 | 80 | 11 | 95 | 13 | 95 | 11 | 85 | 7 |
| B4 | 95 | 10 | 95 | 13 | 35* | 4 | 85 | 6 |

^{a}L = derived from lymph; S = derived from spleen; B = derived from blood.
^{b}Percentage of full-length clones
^{c}Number of different fingerprint patterns (from a total of 20 clones)
*Marked libraries are not included in the final library due to poor insert ratios

Library Selections.

For the initial evaluation of the naive camel library, selections were performed with human Chorionic Gonadotropin (hCG), human Salivary Amylase (hSA) and Arthromyces ramosus Peroxidase (ARP) as antigens. In the blood and lymph samples used for RNA isolations from each camel approximately 10⁷ active B-lymphocytes will be present. Therefore, we inoculated 10⁸ individual TG1 transformants from each sub-library to ensure the presence of the complete naive repertoire of each camel. All sub-libraries, which have passed the quality control check described above, were pooled. For blood and lymph libraries we only corrected for OD600 of the glycerol stocks as all libraries were >10⁷. For the spleen derived libraries we also corrected for the size of the sub-libraries because of the higher expected number of different B-lymphocytes that can be isolated from this tissue. Phages from the A and the B route derived naive libraries were produced as described in the Materials and Methods section, keeping the blood/lymph and spleen derived libraries separate, and used for a first round of selection on the chosen antigens. For all antigens, selections were performed in solution with magnetic beads coated with streptavidin. For ARP, selections in immunotubes were also performed. For soluble selections, antigens were biotinylated as described in Materials and Methods.

In the polyclonal phage ELISA the overall enrichment in each round of selection can be determined. Selection rounds of the naive antibody fragment library constructed via the A route showed only low titres for the antigen of interest and very high titres for streptavidin, which is used in these soluble selections at high concentrations. In selection rounds with the naive antibody fragment library constructed via the B route, the high background titres for streptavidin were not seen. From the two B-route libraries only the library derived from spleen gave high titres for hSA. From these results we decided to test expressed soluble antibody fragments from the selections with the B-route library derived from spleen. From each round of selection 24 single, phage infected, TG1 colonies were inoculated in 2TY medium and propagated in the presence of IPTG for expression of soluble antibody fragments. The number of clones producing an hSA specific antibody fragment after each round of selection was calculated as a percentage of the total number of clones tested.

In round two of this selection the first hSA specific antibody fragments were present. 39% of all clones were specific for hSA. This percentage of clones expressing a specific antibody fragment increased after the third round of selection to 75%.

The selections for hCG specificity were performed as described for hSA. In round 1 we used 100 nM hCG followed by 35 nM in round 2 and 12.5 nM in round 3.

As described for the hSA selections above, polyclonal phage ELISAs were performed prior to the analysis of soluble antibody fragments. In selection with both A-route antibody fragment libraries very high titres were obtained against streptavidin while the titres against hCG remained low. This is the same result as was found in the selections with hSA. The titres of the B-route libraries revealed a much lower streptavidin problem. In the B-route library obtained from blood and lymph the most promising titers were found as almost no background binding with streptavidin was detectable. Individual clones from selections with this B-route library were isolated after each round of selection and their immunogenicity was tested in maxi-sorb ELISA plates coated with 8 μg/ml hCG. The amount of clones producing a hCG-biotin specific antibody fragment after each round of selection was calculated as a percentage of the total amount of antibody fragments tested. After the second round of selection, 23% of the tested individual clones were specific for hCG and after the third round this percentage was 29%.

Selections of antibody fragments specific for the enzyme ARP were performed on the combined A and B route VHH library. Lymph and blood derived libraries were kept separate from the spleen derived libraries. For this antigen, selections with immobilised ARP as well as with soluble biotinylated ARP were performed. In the panning (selection with immobilised antigen) the ARP was coated onto immuno-tubes at concentrations of 113, 35 and 12.5 μg/ml in respective rounds of selections. The prepared phages (see above) were applied onto maxi-sorb tubes coated with 113 μg ARP. After a 2 hour incubation of the phages in these coated tubes in PBS, the tubes were washed. Subsequently, the remaining phages were eluted from the tubes and used to infect E. coli TG1 cells. This procedure was repeated for selection rounds 2 and 3.

Specific ARP antibody fragments were isolated after the third round of selection.

Selections were also performed with Fc domains from IgG and with different Pseudomonas species (P. aeruginosa, P. putida, P. cepacia) as antigen. For each we have isolated specific high affinity antibody fragments (data not shown).

On- and off-rates of a number of hCG and ARP specific antibody fragments were determined by BIACORE analysis. The selected antibody fragments had affinities ranging from 10 to 100 nM.

Discussion

We have used two different methods (route A and the route B amplification strategies) to obtain a naive camel derived library. In route A, the naive VHH repertoire is amplified in a single PCR reaction using the framework 1 region at the 5′ end and the long and the short hinge sequence at the 3′ end for priming. Because it is not known if all hinge sequences present in the camel are known, we also followed a hinge-independent VHH amplification strategy, route B. In this route a primary PCR was been performed using primers in the framework 1 region at the 5′ end and in the CH2 domain at the 3′ end of the fragments. The resulting PCR fragments were then used as a template in a second PCR using primers in the framework 1 region (including a SfiI restriction site) and in the framework 4 region at the 3′ end. Thus via this strategy, we became independent of the hinge region and were able to use two restriction enzymes for cloning which both need eight base pairs for recognition and digestion. In route A, the Sfl restriction site cannot be introduced and therefore we have to use the PstI enzyme which requires only six base pairs for recognition and digestion. Based on the probability of the random occurrence of the PstI sequence, this means that approximately 10% of the PCR fragments will be lost from the library by using a “six-cutter” instead of the “eight-cutter”.

During the performed selections with both the A and the B route libraries, it became clear that specific antibody fragments can be isolated from both libraries.

The DNA sequence of eight selected VHH fragments was determined and the presence of an EcoRV restriction site in the c-myc encoding region revealed that each originated from the naive camel derived VHH library. Analysis of these VHH fragments on a protein level showed that the majority of the fragments contained a serine residue on position 11 (six out of eight fragments). Furthermore, for each specificity, hSA, hCG and ARP, a VHH fragment was identified with a second disulphide bridge between CDRs I and II or III. This demonstrates that the majority of the isolated VHH fragments contain specific camel associated features.

In summary we have constructed a new naive camel derived heavy chain antibody fragment library. PCR and fingerprint analysis of this libraries have shown the high technical quality of the library. Furthermore, selections performed with five antigens resulted in the isolation of specific antibody fragments for each antigen. This new library together with the new naive llama derived library will be a valuable tool for the easy and fast excess of specific high affinity antibody fragments.

The various features and embodiments of the present invention, referred to in individual sections above apply, as appropriate, to other sections, mutatis mutandis. Consequently features specified in one section may be combined with features specified in other sections, as appropriate.

All publications mentioned in the above specification are herein incorporated by reference. Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

1. A method for selecting one or more antibody fragments derived from a non-immunised source having binding specificity for a target antigen, the method comprising
(i) screening an expression library with the target antigen, the library comprising a plurality of nucleic acid sequences cloned from a non-immunised source, each nucleic acid sequence encoding at least part of a variable domain of a heavy chain derived from an immunoglobulin naturally devoid of light chains, the plurality of nucleic acid sequences comprising at least 10⁷ different sequences;
(ii) selecting one or more antibody fragments having the desired specificity for the target antigen; and optionally
(iii) recovering the one or more antibody fragments having the desired binding specificity.

2. The method according to claim 1 wherein the one or more antibody fragments have a binding affinity (K_{d}) of less than 200 nM.

3. The method according to claim 1 wherein the plurality of nucleic acid sequences is derived from lymphoid cells.

4. The method according to claim 1 wherein the plurality of nucleic acid sequences is derived from cDNA clones.

5. The method according to claim 1 wherein the at least part of the variable domain of a heavy chain is derived from a camelid immunoglobulin.

6. The method according to claim 3 wherein the at least part of the variable domain of a heavy chain is derived from a camelid immunoglobulin.

7. The method according to claim 1 wherein the antibody fragments encoded by the plurality of nucleic acid sequences are displayed on the surface of bacteriophage.

8. The method according to claim 1 wherein the plurality of nucleic acid sequences comprises at least 108 different sequences.

9. The method according to claim 1 which further comprises a step (iv) of isolating the nucleic acid sequence(s) encoding the selected one or more antibody fragments.

10. A method for preparing an antibody derived from a non-immunised source having binding specificity for a target antigen, the method comprising
(i) isolating a nucleic acid sequence encoding an antibody fragment having the desired binding specificity for the target antigen by the method of claim 8; and
(ii) operably linking the region of the nucleic acid sequence encoding at least part of a variable domain of a heavy chain derived from an immunoglobulin naturally devoid of light chains to one or more nucleic acid sequences encoding one or more heavy chain constant domains; and
(iii) expressing the resulting product in a host cell.

11. An expression library comprising a plurality of nucleic acid sequences cloned from a non-immunised source, each nucleic acid sequence encoding at least part of a variable domain of a heavy chain derived from an immunoglobulin naturally devoid of light chains, the plurality of nucleic acid sequences comprising at least 10⁷ different sequences.

12. An expression library according to claim 11 comprising at least 10⁸ different sequences.

13. The library according to claim 11 wherein the plurality of nucleic acid sequences is derived from lymphoid cells.

14. The library according to claim 11 wherein the plurality of nucleic acid sequences is derived from cDNA clones.

15. The library according to claim 11 wherein the at least part of the variable domain of a heavy chain is derived from a camelid immunoglobulin.


SEQUENCE LISTING (18 sequences)

SEQ ID NO: 1 - DNA, 22 bp, Artificial Sequence
  Description of Artificial Sequence Primer
aggtsmarctgcagsagtcwgg

SEQ ID NO: 2 - DNA, 57 bp, Artificial Sequence
  Description of Artificial Sequence Primer
catgccatgactcgcggcccagccggccatggccsaggtsmarctgcagsagtcwgg

SEQ ID NO: 3 - DNA, 53 bp, Artificial Sequence
  Description of Artificial Sequence Primer
aacagttaagcttccgcttgcggccgcggagctggggtcttcgctgtggtgcg

SEQ ID NO: 4 - DNA, 53 bp, Artificial Sequence
  Description of Artificial Sequence Primer
aacagttaagcttccgcttgcggccgctggttgtggttttggtgtcttgggtt

SEQ ID NO: 5 - protein, 117 aa, Lama glama
QVQLQESGGGLVQAGDFLRFSCAALGARFSSDVMGWFRQAPGKEREFVAASSWNGDTTHYSDSVEGQFTISRDIAKNTSYLQMNRLQPEDTAVYYCRWCRPPRPKYWGQGTQVTVSS

SEQ ID NO: 6 - protein, 115 aa, Lama glama
QVQLQQSGGGLVQAGSFLSFSCTASGRTFSNYAMGWFRQASGNQRAFVAAIGRNGDTHYIDSVKGRFTISRDNGKDTVYLQMNSLKPEDTAVYYCRIWVGARDYWGQGTQVTVSS

SEQ ID NO: 7 - protein, 116 aa, Lama glama
QVQLQESGGGLVQAGGFLRFSCAASGRTFSRYTMGWFRQAPGNERKFVAAVSTSGNTHYTGSVKGRFTIFRQNAKNTVYLQMSNLKPEDTAVYYCAARFGGMNWKYWGQGIQVTVS

SEQ ID NO: 8 - protein, 121 aa, Lama glama
QVQLQESGGGLVQPGPFLNVSCVVSGGIFSDYTLGWFRQAPGKERKFVAAVSSGGSTHYTGSVKGRFTISRDNAANTMYLQMSSLKPDDTAVYYCNAIVPPTRTFCGRTYWGQGTQVTVSS

SEQ ID NO: 9 - protein, 112 aa, Lama glama
QVQLQESGGGLVQPGDFVRLSCAASRRASSTYAVGWFRQAPGKEREFVGRIHRGGGSTYYADSVKGRFTISRDNTQNTVYLQMNSLKPEDTAVYYCNVRSYWGQGTQVTVSS

SEQ ID NO: 10 - protein, 117 aa, Lama glama
QVQLQESGGGLVQAGGFLRFSCAASNALFSGYAMGCFRQAVGKEREFVAAITWNNRNTHYADSVKGRFTISRDNAKNTVYLQMNSLKPEDTAVYYCTSGMRRLGDYWGQGTQVTVSS

SEQ ID NO: 11 - protein, 124 aa, Lama glama
QVKLQESGGGLVQAGGSLRLSCAASGFTFDKYAIGWFRQAPGKQRELVAGISTGGSTNYADSVKGRFTISRDNAKDTVYLQMNSLKPEDTAVYYCAAGRRISSSYYSRGLYAYWGQGTQVTVSS

SEQ ID NO: 12 - protein, 124 aa, Lama glama
QVQLQESGGGLVQAGDSLRLSCEASGRSFSNFAMAWFRQTPGKEREFVAGISWRGGRTYYAASVKGRFTISRDNGKNTVYLQMNSLKPEDTAVYYCATAYGQGPITVPKFYTYRGQGTQVTVSS

SEQ ID NO: 13 - protein, 121 aa, Lama glama
QVQLQESGGGLVQAGGCVRLSCAASGRTFSRYTMGWFRQAPGKEREFVAAISWRSGGIKIYGDSVKGRFTISRDNAKDTVYVQMNSLKPEDTAVYYCNSRPRIYRGNVVYWGQGTQVTVSS

SEQ ID NO: 14 - DNA, 34 bp, Artificial Sequence
  Description of Artificial Sequence Synthetic oligonucleotide
ggcccagccggccatggcccaggtgcagctgcag

SEQ ID NO: 15 - protein, 11 aa, Artificial Sequence
  Description of Artificial Sequence Synthetic peptide
AQPAMAQVQLQ

SEQ ID NO: 16 - DNA, 39 bp, Artificial Sequence
  Description of Artificial Sequence Synthetic oligonucleotide
gcggccgcccatcaccatcaccatcacggggccgcagaa

SEQ ID NO: 17 - protein, 13 aa, Artificial Sequence
  Description of Artificial Sequence Synthetic peptide
AAAHHHHHHGAAE

SEQ ID NO: 18 - protein, 11 aa, Unknown Organism
  Description of Unknown Organism Myc peptide sequence
EQKLISEEDLN